(19) United States
(12) Patent Application Publication
(10) Pub. No.: US 2008/0057001 A1
(43) Pub. Date: Mar. 6, 2008

(54) CONTRAST AGENTS FOR IMAGING

(76) Inventor: Xiao-Dong Sun, Fremont, CA (US)

Correspondence Address: SHERR & NOURSE, PLLC, 620 HERNDON PARKWAY, SUITE 200, HERNDON, VA, 20170, US

(21) Appl. No.: 11/751,732

(22) Filed: May 22, 2007

Related U.S. Application Data
(60) Provisional application No. 60/808,257, filed on May 25, 2006.
(60) Provisional application No. 60/819,667, filed on Jul. 10, 2006.

(51) Int. Cl. A61K 49/18

(52) U.S. Cl. 424/9.322; 424/9.32

(57) ABSTRACT

Contrast agents and methods for making them are presented that use Fe nanoparticles that produce higher clarity and particularity in MRI imaging. Various alloys and core compounds are presented that may be used to produce such higher clarity MRI images.

This application claims priority to U.S. Provisional Patent Application Ser. Nos. 60/808,257 (filed May 25, 2006) and 60/819,667 (filed Jul. 10, 2006), the content of which is hereby incorporated by reference in its entirety into this disclosure.

### BACKGROUND OF THE INVENTION

1. Field of the Invention

The present invention relates to contrast agents. In particular, the present invention relates to contrast agents used for magnetic resonance imaging (MRI).

2. Background of the Invention

One of the most powerful and non-invasive tools that clinicians utilize in order to gain further insight into the structural or physiological functions or changes within a body is magnetic resonance imaging (MRI). This tool allows a specialized magnetic reader to detect and measure proton relaxation signals that can be varied by contrast agents localized within an area of interest that have been pre-digested or pre-injected into the body. Such contrast agents are a key component of determining the ultimate sensitivity of the MRI image.

In general, contrast agents are chemical substances introduced into the anatomical or functional region being imaged, to increase the differences between different tissues or between normal and abnormal tissue, by altering the relaxation times for the image. MRI contrast agents are generally classified by the different changes in relaxation times after their injection. There are currently two general classifications of MRI agents, positive and negative agents.

Positive contrast agents cause a reduction in the T1 relaxation time. In other words, they have increased signal intensity on T1 weighted images. Thus, they appear bright on an MRI image. They are typically small molecular weight compounds often containing as their active element a rare earth molecule such as gadolinium or manganese. All of these elements have unpaired electron spins in their outer shells and long relaxivities.

Negative contrast agents, in contrast to positive contrast agents, appear dark on MRI images. They are small particulate aggregates often termed superparamagnetic iron oxide (SPIO). These agents produce predominantly spin-spin relaxation effects (local field inhomogeneities), which results in shorter T1 and T2 relaxation times. SPIO's and ultrasmall superparamagnetic iron oxides (USPIO) usually include a crystalline iron oxide core containing thousands of iron atoms and a shell of polymer, dextran, polyethyleneglycol, and produce very high T2 relaxivities.

Despite these different classes of contrast agents, a major remaining issue of MRI is still the lack of sensitivity. Dispersions of magnetic nanoparticles have been used as contrast agents for MRI due to their large magnetic moment that enhances the relaxation rates of proton in specific organs. For example, negative contrast agents, such as commercially available Feridex I.V.™ and Resovist, which are based on SPIO nanoparticles, are conventionally used in MRI procedures. A typical structure of such negative contrast agent comprises of a magnetic core (e.g., Fe₃O₄) and a polymer coating (e.g., dextran, PEG). However, such conventional contrast agents still do not produce images to the degree of clarity often demanded by clinicians.

Despite the advances that MRI has brought to the clinical setting, the full potential of this powerful new instrument has been limited by the functionality of the contrast agents used. Thus, there is a need in the art for a more effective and sensitive contrast agent that allows for sharper, clearer and more robust MRI images without suffering from some of the drawbacks of conventional contrast agents. The contrast agent should be simple to produce and administer, effective and capable of producing consistently high quality MRI images.

### SUMMARY OF THE INVENTION

The present invention presents a new class of contrast agents that produce higher quality and accuracy MRI images than conventional contrast agents. Furthermore, the present invention provides for methods of producing these novel types of contrast agents. The discovery of such novel contrast agents was based on the notion that in the exploration of highly-sensitive MRI contrast agent, more effective magnetic cores are the key in improving the contrast and sensitivity of the agents.

It is expected that contrast agents containing magnetic cores of higher saturation magnetization and magnetic susceptibility values can further enhance relaxation rates of protons at a significantly lower dose and improve contrast. While iron oxide (Fe₃O₄) is a good ferromagnetic material, the present invention is based on the discovery that there are other magnetic materials with better properties.

In one exemplary embodiment, the present invention is nano-particles of iron, or alloys of iron with Carbon, Cobalt, that can be applied as MRI contrast agents, with particle sizes smaller than 1000 nm, more preferably below 100 nm

In another exemplary embodiment, the present invention is a core-shell structure of nano-particles, with Fe core in the size of <1000 nm, or preferably <100 nm, and a shell of Carbon, Cobalt, SiOx, and Gold

In yet another exemplary embodiment, the present invention is coating of polymers, and/or organic molecules including drugs or other binding/inhibiting agents, onto the aforementioned magnetic nano-particles in the previous embodiments. Polymer coatings, such as Dextran, PEG, Starch, etc., help enhance the biocompatibility of the nano-particles.

### BRIEF DESCRIPTION OF THE DRAWINGS

FIG. 1 shows a magnetic hysteresis curve of Fe nanoparticles (10 nm) at room temperature, according to an exemplary embodiment of the present invention.

FIG. 2 shows a magnetic hysteresis curve of Fe nanoparticles (26 nm) at room temperature, according to an exemplary embodiment of the present invention.

FIG. 3 shows a magnetic hysteresis curve of Fe/C nanoparticles (25 nm) at room temperature, according to an exemplary embodiment of the present invention.

FIG. 4A shows a magnetic hysteresis curve of iron nano-particle (10 nm), according to an exemplary embodiment of the present invention, and SPIO magnetite (Fe₃O₄ 30 nm) nanoparticles.

FIG. 4B shows a magnetic hysteresis curve of dextran-coated metallic iron (10 nm) nanoparticle ferrofluid with a noted lack of hysteresis in the coated sample, according to an exemplary embodiment of the present invention.

FIG. 5 shows a saturation magnetization of Y₃Fe₅O₁₂ garnet at various particle sizes, according to an exemplary embodiment of the present invention.

FIG. 6 shows saturation induction versus coercivity for commercially available amorphous metals (AM) and crystalline soft ferromagnets.

FIG. 7 shows a chemical vapor deposition (CVD) system to prepare magnetic nano-particles of various sizes and compositions, according to an exemplary embodiment of the present invention.

FIG. 8 shows a diagram of nanoparticle surface modification with hydrophilic polymers, such as PEG and starch derivatives, according to an exemplary embodiment of the present invention.

FIGS. 9A-9H show SEM/TEM images of various produced particles according to exemplary embodiments of the present invention.

### DETAILED DESCRIPTION OF THE INVENTION

This invention describes techniques for obtaining more enhanced MRI images by producing a novel class of contrast agents, and the methods for producing such novel agents.

The present invention shows that the saturation magnetization, which is a conventional test of the effectiveness of a contrast agent, depends on both the particle size and the compositions of the nanoparticles within a core of the contrast agent vehicle. Thus, the present invention sought to produce a class of negative contrast agents with cores having much higher sensitivities than conventional negative contrast agents. Although the outer shell of the negative contrast agent according to the present invention may be relatively similar to that of the outer shell of conventional negative contrast agents in certain exemplary embodiments, the core is significantly different.

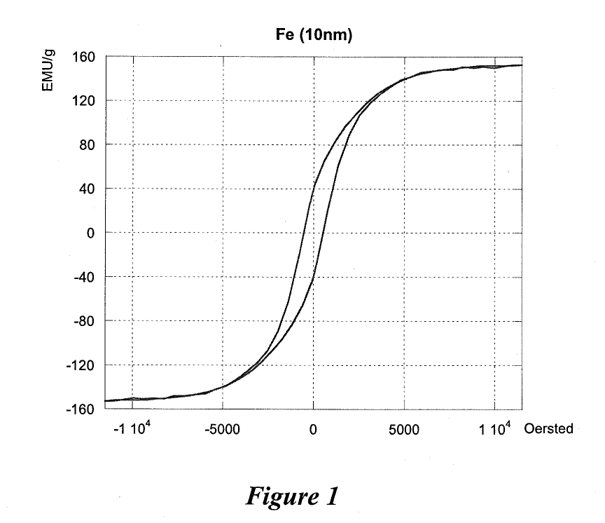

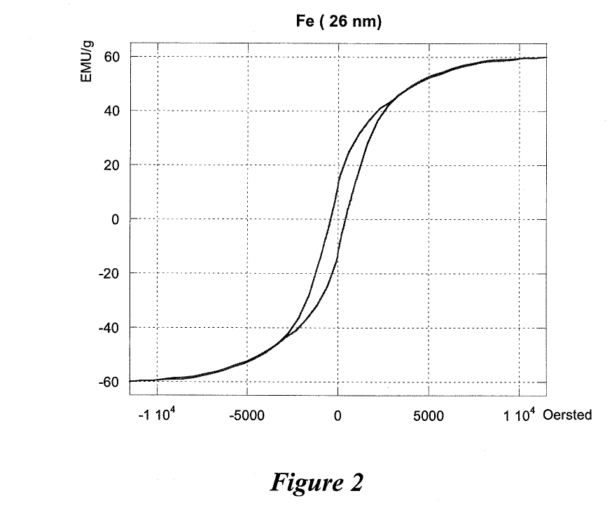

In one exemplary embodiment, iron nanoparticles according to the present invention and produced with average sizes of 10 nm and 26 nm, respectively, were found to have saturation magnetization of 152.5 emu/g Fe, and 60.0 eum/g Fe, respectively, as shown in FIG. 1 and FIG. 2. In comparison, conventional contrast agents with iron oxide based nanoparticles (e.g., Feridex I.V.) had a saturation magnetization of ˜68 emu/g Fe. As a result, evidence shows that the metallic iron nano-particles with particle sizes of ˜10 nm or less can serve as a highly sensitive core of advanced MRI contrast agents. The pure iron nano-core can be encapsulated by polymers, other alloys, or ceramics, to prepare MRI contrast agents. The samples in FIGS. 1 and 2 are measured in solid powder form, and there is some small magnetic hysteresis. Once the nano-particles are dispersed in a liquid and form a colloid or suspension, the hysterisis will be reduced or eliminated to show a superparamagnetic property.

Furthermore, as shown in FIG. 3, it was discovered that iron nanoparticles coated with carbon (Fe/C, ˜25 nm) have a high saturation magnetization of 119 emu/g, which is much higher than the pure metallic iron of similar size (see FIG. 2, 26 nm). Thus, this provides support to the finding that Fe/C with sizes 10 nm or less have even higher saturation magnetization than pure iron nanoparticles, which will be an even better core for MRI contrast agent in improving the sensitivity of the contrast agent. The magnetic properties of iron and iron/carbon nanoparticles are shown in Table 1.

TABLE 1
Magnetic properties of iron and iron/carbon nanoparticles

| | | Saturation | Remanent |
|---|---|---|---|
| | Coercivity | Magnetization | Magnetization |
| Material | (Oe) | (EMU/g) | (EMU/g) |
| Fe (10 nm) | 520 | 152.5 | 40 |
| Fe (26 nm) | 400 | 60.0 | 13 |
| Fe/C (25 nm) | 190 | 119.0 | 15 |

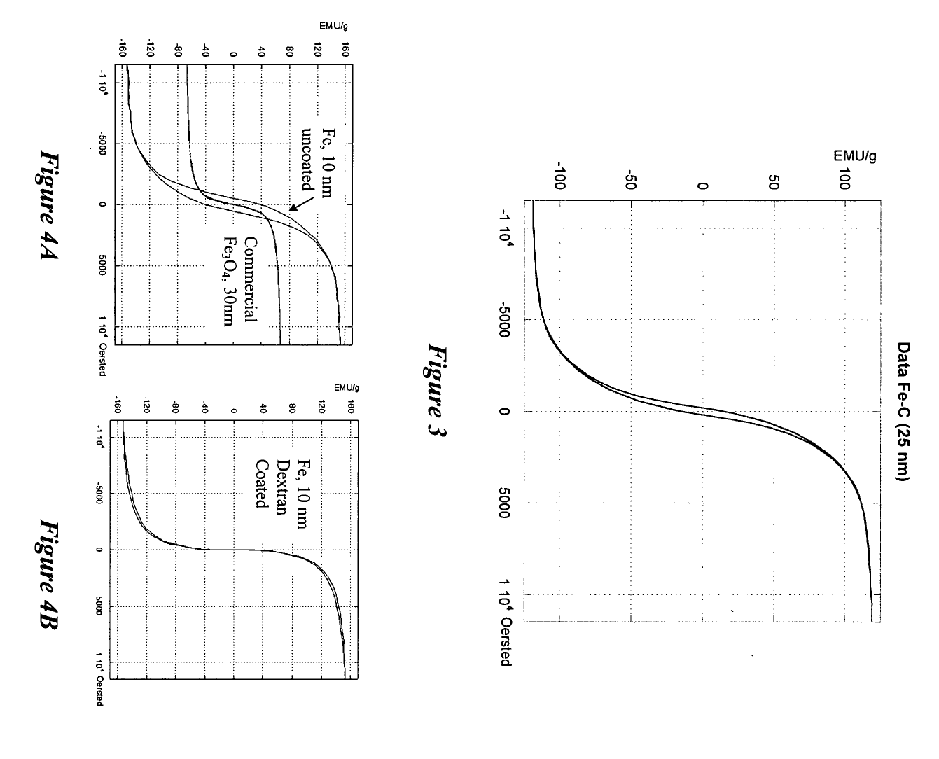

Studies resulted in synthesis and evaluation of the nano-magnets on advanced magnets. In one exemplary embodiment, magnetite nanoparticles (Fe₃O₄) were prepared using a sonochemical method. The nano-particles have an average particle size of 30 nm. The saturation magnetization from the M-H measurement for the magnetite nanoparticles (Fe₃O₄) is 67 emu/g (FIG. 4a), which is significantly higher than that of commercial iron oxide nano-particles prepared by co-precipitation (˜51 emu/g). Given the significant higher bulk magnetization of Fe over Fe₃O₄ (FIG. 4A), iron nanoparticles were prepared of two different particle sizes: 10 nm and 26 nm. The method of the synthesis is presented in further detail below. The saturation magnetization of iron nanoparticles of 26 nm was found to be ˜60 eum/g. Counter intuitively, it was found that when iron particle size was reduced to 10 nm, the saturation magnetization value was actually increased dramatically to 152.5 eum/g (FIG. 4A), which is 2.4 times higher than iron nanoparticles of 26 nm prepared by the same method, and about 3 times that of commercial Fe₃O₄ nanoparticles prepared by co-precipitation.

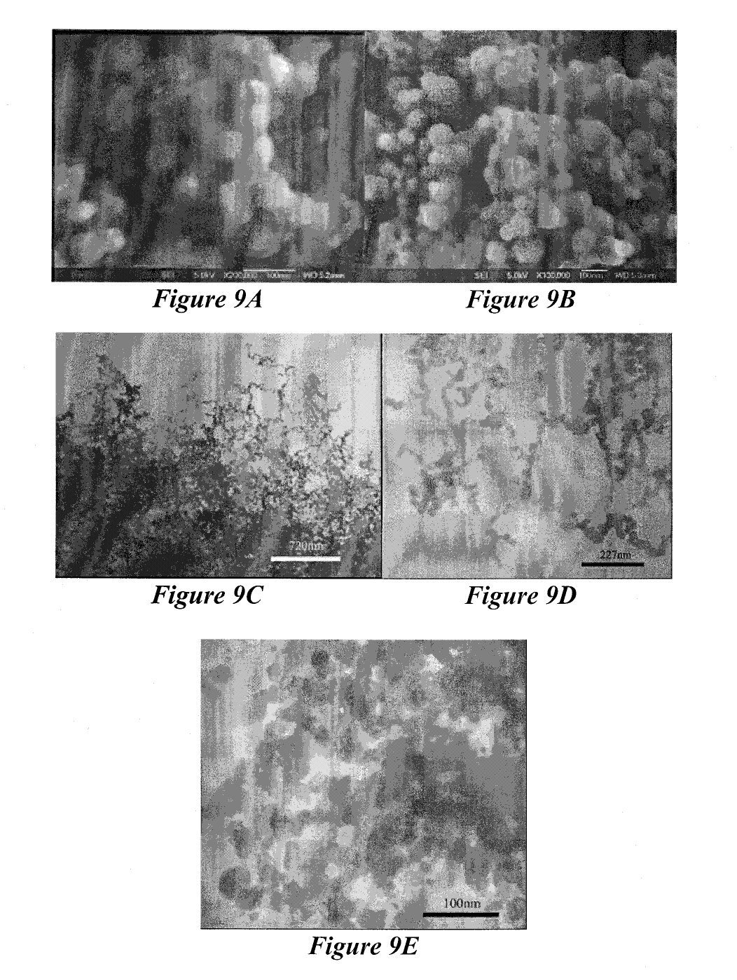

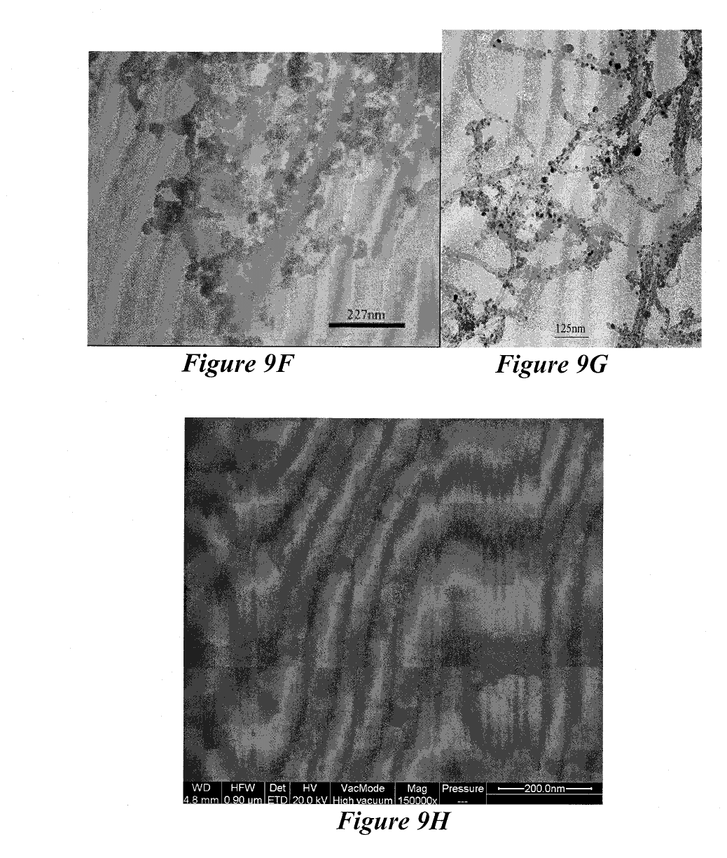

Iron metal nanoparticles (10 nm) were coated with dextran polymer to make stable colloidal suspension. SEM micrographs of non-coated (left) and dextran-coated (right) iron nanoparticles are shown in FIGS. 9A and 9B. The dextran-coated iron nanoparticles have similar magnetization to non-coated nanoparticles but without remanence magnetization or hysteresis, showing the dispersed iron nano-particles are indeed superparamagnetic. The magnetic curve of dextran-coated iron nanoparticle (10 nm) colloid is shown in FIG. 4B. While the dextran coated and the uncoated Fe nano-particle (FIG. 4A) have similar saturation magnetization (˜160 emu/g), the coated nano-particle has improved magnetic susceptibility due to lack of magnetic hysteresis. A very stable aqueous colloidal solution has been obtained with Fe nano-particles after the dextran coating, with Fe concentration of ˜13 mg/ml.

In addition to simple magnetic metals or their carbon alloys, other possible good candidates for magnetic cores include metallic alloys, such as, for example, FeₓCo₁₋ₓ, 1>x>0. The Fe—Co alloy has ever higher saturation magnetization than pure iron at certain range of compositions (e.g., 0.3≦x≦1). Such Co—Fe alloy can form a magnetic core of advanced MRI contrast agent. Some other elements (e.g., carbon, vanadium, chromium) may be added to the Co—Fe alloy system to enhance other properties. While Fe can alloy with other elements such as Si, Al, Ni, Cu, Mn, Cr, etc., their saturation magnetization is generally less than pure iron. Nevertheless, such alloy can still be used as the magnetic core of MRI contrasting agent.

In addition to forming alloys, a core-shell structure of metal nano-particles, with a core of Fe, FeOₓ, Co, Ni, magnetic ferrites, Nd—Fe—B based magnets or other ferromagnetic compositions, with a diameter less than or equal to about 1000 nm, or preferably less than or equal to about 100 nm, more preferably less than or equal to about 20 nm, or even more preferably less than or equal to about 10 nm; and a shell of Fe, Co, Ni, Gold, Carbon, SiOₓ, Cu, Mn, Cr, V, Ag, Al, etc. Shells with other physical, chemical, or biological functions can also be applied to the nano-magnetic cores. Such core shell structures results in nano-magnets with either enhanced magnetic properties, or combination of magnetic properties with other physical, chemical, and biological properties of interests. They can be used for MRI contrast agents, as well as many other applications, including bio-sensing, diagnosis and therapy.

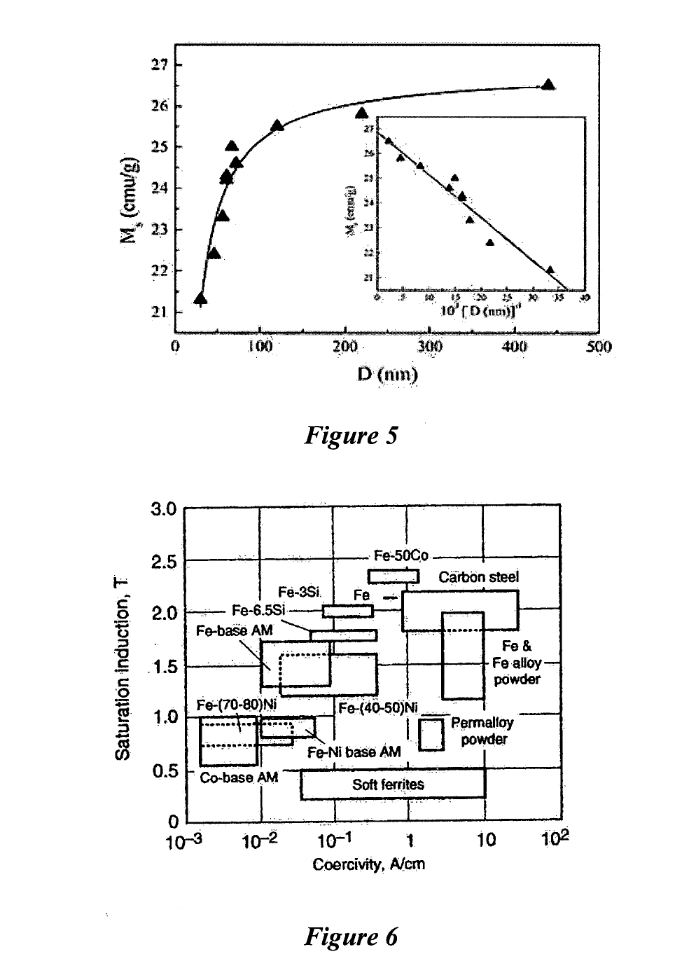

Finally, there are a few other magnetic oxides, with magnetic properties similar or better than iron oxide (Fe₃O₄). A few examples of such magnetic oxides include other ferrites, magnetic garnets, etc. More specifically, they include Y₃Fe₅O₁₂; Nickel-Zinc Ferrite, or Ni₁₋ₓZnₓFe₂O₄; BaFe₁₂O₁₉, and BaFe₁₈O₂₇, etc. An exemplary hysteresis graph of the Y₃Fe₅O₁₂ garnet is presented in FIG. 5. The applicable compositions of the magnet cores for MRI contrasting agent, however, are not limited to these few specific compositions. Other ferrites or magnetic garnets with different combinations or substitution of elements with iron could also be used as the core of MRI contrast agents.

A graphical depiction of various compounds that may be used for a core according to the present invention is shown in FIG. 6. Compounds shown higher on the graph, such as Fe, are those with strongest known bulk magnetization, and thus, better to use as a core material in MRI contrast agents. Those shown lower on the graph, such as soft ferrites, are conventionally used compounds in core material of MRI contrast agents. Thus, it would be beneficial to use compounds presented higher on this graph in producing core material according to the present invention.

Various methods may be utilized to produce one or more of the agents described above and in accordance to the present invention. In one exemplary embodiment in producing a dispersion using dextran, an iron-dextran colloidal dispersion may be produced. In such process, a suspension of 200 mg of iron nanoparticles in 2.5 mL of NaOH (0.5 M) was prepared by sonication for 5 min. It was added slowly to a solution of 200 mg of dextran 5 kDa in 2.5 mL NaOH (0.5 M), used as dispersant plus coating medium, under sonication (addition time 30 min). The sonication was kept for 24 h at 30° C. in order to favor dispersion of the iron nanoparticles and the link of dextran chain on its surface. The sonication process is carried out in an ultrasonic bath provided with a refrigeration coil to avoid overheating at 35 W and 35 KHz, the suspension was held in a standard 25 mL test tube of wall thickness 0.5 mm. Then, the dispersion will be dialyzed for 24 h in 5 L of distilled water using a 12,000-14,000 nominal cut off molecular weight membrane. Tri-sodium citrate dehydrated (4 mg, ˜1 mM) and L-mannitol (0.60 g, ˜5 wt %) was added in order to make the suspension suitable for parental administration. Finally, the resulting stable magnetic dispersion will be refined and made sterile by filtration through a 0.1 μm pore size filter. The iron concentration in the colloidal suspensions was measurement by total reflection X-ray fluorescence (TXRF), using a Seifert Extra-II spectrometer and cobalt as internal pattern for the calibration.

SEM was employed to examine the aggregation of the particles in the suspension. The mean hydrodynamic diameter of the aggregates, corresponding to the magnetic particles plus to the dextran coating, was determined by photon correlation spectroscopy (PCS) in a Zetasizer 1000 HS, Malvern Instruments. The peak analysis in the volume was made using the method of cumulants. A log-normal distribution function was used to fit the size data obtained from the different techniques.

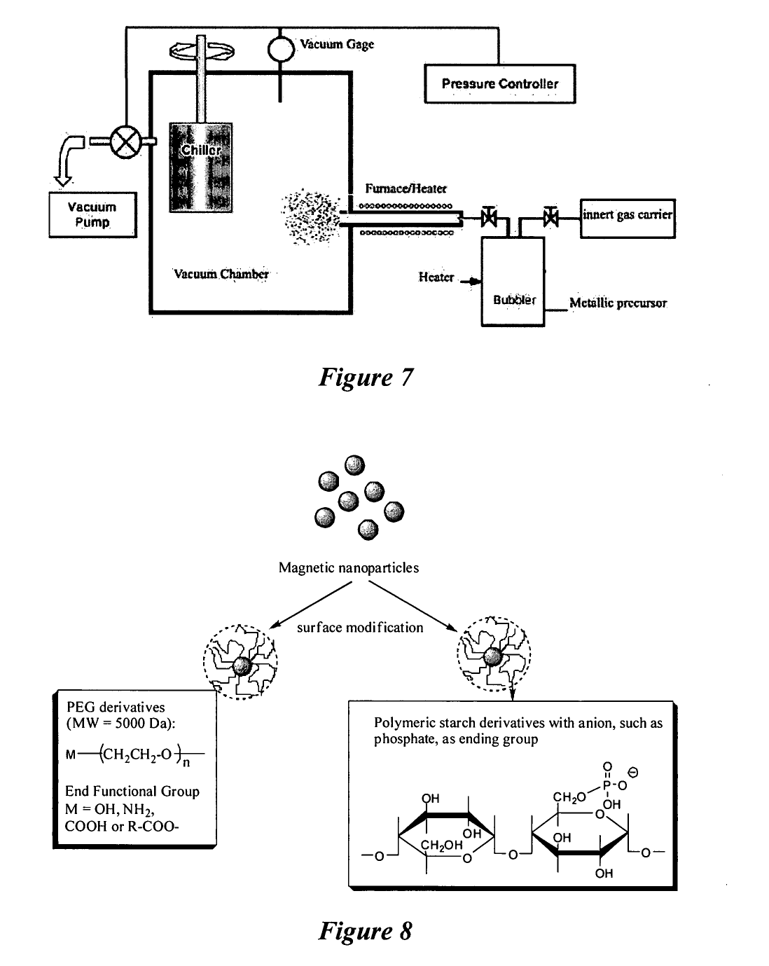

In another exemplary embodiment, a PEG or starch method is used. In this method, a preparation and characterization of Fe—Co magnetic nanoparticles are described. A novel chemical vapor deposition (CVD) method is used to prepare Fe—Co based magnetic nanoparticles with small particles sizes (5 to 50 nm), using the organometallic precursors. Iron/Cobalt nanoparticles of various sizes and compositions are prepared using a CVD reactor, as shown in FIG. 7. Carrier gas (e.g., Helium) may be applied to a precursor bubbler and carry the organometallic vapor to a horizontal furnace. The vapors react and decompose into atomic clusters and condense onto the chiller in a vacuum chamber. Particle sizes and compositions can be varied by adjusting the relative partial pressures of the various gas reactants through bubblers. The synthesized powders can be scalped off and collected from a rotating chiller cooled by liquid nitrogen.

Iron Cobalt (Fe—Co) nano-alloys can be synthesized using iron pentacarbonyl [Fe(CO)₅] and cobalt octacarbonyl [Co₂(CO)₈] as precursors. The flow rate of the carrier gas can be varied to change the relative composition of the Fe:Co feedstock in the vapor, resulting in nano-clusters with various Fe—Co alloy compositions. The temperatures of the furnace can vary from 600 to 1200° C., to synthesize nano-particles of different particle sizes. The total metallic concentration of organo-metallic vapor can also be varied to control the particle sizes. In order to prevent the explosion, a small amount of air may be supplied into the chamber during the cooling.

Nano-magnet Fe—Co particles of various compositions are prepared and made with particle sizes between 5 and 50 nm. The elemental compositions and sizes of the nano-particles synthesized and investigated are summarized in the following Table 2. A total of 16 different nano-particles covering 4 different Fe—Co composition and 4 different particle sizes for each composition are synthesized and studied, for superior nano-magnet for MRI contrast agent. From such experimental matrix, the relationship of magnetic properties (e.g., saturation magnetization and susceptibility) with the elemental compositions and particle sizes can be derived. The optimum size and composition for the most desirable magnetic properties can be calculated.

TABLE 2
Fe—Co based Nano-Magnets with targeted compositions and sizes to
be prepared and studied in Phase I project

| | Composition | | | |
|---|---|---|---|---|
| | FeCo_{0.25} | FeCo_{0.5} | FeCo_{0.75} | FeCo |
| Sizes | 5, 15, 30, | 5, 15, 30, 50 | 5, 15, 30, 50 | 5, 15, 30, 50 |
| (nm, ±2 nm) | 50 | | | |

The as-prepared nanoparticles are further characterized by TEM, XRD and vibrating sample magnetometer to obtain structural morphology, size and magnetic properties.

In further developing the contrast agent, surface chemistry on the magnetic nano-particles may also be changed according to the present invention. The magnetic nanoparticles used in biomedical applications need special surface modifications that are non-toxic and biocompatible. The surface chemistry of the magnetic particles strongly affects both the blood circulation time and bioavailability of the particles within the body. To stabilize the magnetic nano-particles in aqueous solution (e.g., blood) and increase biocompatibility, these superparamagnetic nanoparticles will be coated with biocompatible hydrophilic polymers, such as PEG derivatives and polymeric starch. The sizes of the coated nanoparticle complexes shall be under 100 nm with overall narrow particle size distribution, which is optimal for intravenous injection. The resulting prolonged circulation time in the blood stream due to hydrophilic surface coating can evade clearance by the reticuloendothelial system.

PEG is widely used as a coating material for nanoparticles in biological research due to uncharged hydrophilic residues and very high surface mobility leading to high steric exclusion. Surfaces covered with PEG are biocompatible, i.e., nonimmunogenic, nonantigenic, and protein-resistant. Therefore, covalently immobilizing PEG on the surfaces of superparamagnetic magnetite nanoparticles is expected to efficiently improve the biocompatibility of the nanoparticles. In addition, PEG has high solubility in cell membranes. It has been demonstrated that particles with PEG-modified surfaces can cross cell membranes in non-specific cellular uptake due to its solubility in both polar and nonpolar solvents. According to Gupta and Wells, PEG “protects surfaces from interacting with cells or proteins. Thus, PEG-coated particles may result in increased blood circulation time”. Starch is a long chain polymer of D-glucose and is abundant naturally as one of the polysaccharides. It has also been chosen as good coating polymers for biomedical applications due to its biocompatibility, biodegradability and nontoxicity. Starch derivatives with functional ending groups (e.g., phosphate) are hydrophilic and allow ionic binding to many therapeutic drugs.

The surface coating of the superparamagnetic nanoparticles is based on polymeric starch and PEG derivatives because of their properties mentioned above. The presence of a polymeric network hinders the agglomeration of the magnetic nanoparticles and holds the particles apart against attracting forces by surface intension and dipole-dipole interaction. Furthermore, the polymer layer on the surface of the particles prevents further oxidation. The schematic diagram of magnetic coating with PEG or starch derivatives are shown in FIG. 8.

FIGS. 9A-9H show various SEM and TEM micrographs of the cores, particles and contrast agents produced according to the present invention. FIGS. 9A and 9B show SEM micrograph of non-coated (left) and dextran-coated (right) iron nanoparticles according to the present invention.

FIGS. 9C, 9D and 9E show agglomeration of Fe particles at 27.5 k×magnification, 88.0 k×magnification, and 200.0 k×magnification, respectively.

FIG. 9F shows clusters of Fe-Carbon at 88.0 k×magnification. FIG. 9G shows TEM images of the Fe—Co alloy nanoparticles attached on carbon nanotubes Co—Fe:Carbon. FIG. 9H shows pure Nano Fe/Co Alloy Powder (Fe:Co=1:1) Average Particle Size: ≈30 nm (by HRTEM) with special surface area: 80-160 m²/g, Magnetic flux density: 1.2 T.

It should be noted that images 9C-9H were obtained using a Philips CM20 transmission electron microscope/scanning transmission electron microscope (TEM/STEM) analytical microscope operated at 200 keV with EDX analytical mapping was also used to collect images and Energy Dispersive X-ray (EDX) spectra from powder scrapings. The powders were taken directly from sample jar to TEM vacuum, exposure to open air was <1 min. The results of the Energy Dispersive X-ray (EDX) showed 100% Fe with no impurities (+/−1%). Particles consisted of single grains. Particle size was calculated to be 26+/−6 nm. This was calculated as an average and standard deviation of 50 particles. Particles appear somewhat nodular with some elongation. Chains formed could be due to the magnetic properties of the particles.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

What is claimed is:

1. A contrast agent for magnetic resonance imaging, the contrast agent comprising:
a metallic magnetic core comprised of Fe nanoparticles less than or equal to about 100 nm; and
a shell containing the core.

2. The contrast agent of claim 1, wherein the nanoparticles are less than or equal to about 20 nm.

3. The contrast agent of claim 2, wherein the nanoparticles are less than or equal to about 10 nm.

4. The contrast agent of claim 1, wherein the shell includes a polymer.

5. The contrast agent of claim 1, wherein the shell includes one or more of the following inorganic substances: Co, Ni, FeAu, C, FeOx, and SiOx.

6. The contrast agent of claim 1, wherein the shell includes one or more of the following substances to make the core biocompatible: dextran, PEG, and starch.

7. The contrast agent of claim 1, wherein the shell adds additional physical, chemical or therapeutic function to the magnetic core.

8. The contrast agent of claim 1, wherein the Fe nanoparticles comprise an alloy.

9. The contrast agent of claim 8, wherein the alloy contains Co or C.

10. A contrast agent for magnetic resonance imaging, the contrast agent comprising:
a core comprised of Fe—Co or Fe—C nanoparticles; and
a shell containing the core.

11. The contrast agent of claim 10, wherein the nanoparticles are less than or equal to about 100 nm.

12. The contrast agent of claim 11, wherein the nanoparticles are less than or equal to about 20 nm.

13. The contrast agent of claim 12, wherein the nanoparticles are less than or equal to about 10 nm.

14. The contrast agent of claim 10, wherein the shell includes a polymer.

15. The contrast agent of claim 10, wherein the shell includes one or more of the following substances to make the core biocompatible: dextran, PEG, and starch.

16. The contrast agent of claim 10, wherein the shell adds additional physical, chemical or therapeutic function to the magnetic core.

17. The contrast agent of claim 10, wherein the saturation magnetization is greater than 100 emu/g Fe.

18. A method of producing a contrast agent for magnetic resonance imaging, the method comprising:
providing a metallic magnetic core comprised of Fe nanoparticles; and
enclosing the core within a shell.

19. The method of claim 18, wherein the Fe nanoparticles are less than or equal to about 100 nm.

20. The method of claim 19, wherein the nanoparticles are less than or equal to about 20 nm.

21. The method of claim 20, wherein the nanoparticles are less than or equal to about 10 nm.

22. The method of claim 18, wherein the Fe nanoparticles comprise an alloy.

23. The method of claim 22, wherein the alloy contains Co or C.

24. The method of claim 18, wherein the enclosing step includes chemical vapor deposition or decomposition.

25. The method of claim 18, wherein the shell includes one or more of the following substances to make the core biocompatible: dextran, PEG, and starch.
